(19) United States
(12) Patent Application Publication
(10) Pub. No.: US 2005/0042720 A1
(43) Pub. Date: Feb. 24, 2005

(54) Novel promiscuous G alpha protein mutants and their use

(75) Inventor: Evi Kostenis, Humlebaek (DK)

Correspondence Address: EDWARDS & ANGELL, LLP, P.O. Box 55874, Boston, MA, 02205, US

(73) Assignee: 7TM Pharma A/S, Horsholm (DK)

(21) Appl. No.: 10/830,716

(22) Filed: Apr. 24, 2004

(30) Foreign Application Priority Data
Apr. 24, 2003 (DK) PA 2003 00624

(51) Int. Cl. C07K 14/705; C07H 21/04; C12N 9/48

(52) U.S. Cl. 435/69.1; 435/320.1; 435/325; 530/350; 536/23.5

(57) ABSTRACT

The present invention provides q-type G proteins, and nucleic acids encoding q-type G proteins, with one or more mutations in the linker I and/or linker II region, which render the proteins responsive to one or more non-q type G protein coupled receptors. The invention further relates to methods of identifying modulators of a G-protein coupled receptor.

### FIELD OF THE INVENTION

The present invention relates to novel mutants of q-type G protein, the mutations rendering the q-type G protein responsive to one or more none type G protein coupled receptors.

### INTRODUCTION

The G protein-coupled receptors (GPCR) constitute the largest family of proteins in the human genome and function as receivers of all kinds of chemical signals. Stimuli as diverse as light, odorants, nucleotides, neurotransmitters and hormones exert their effects via the GPCR-G protein signal transduction cascade. The spectrum of hormones, neurotransmitters, paracrine mediators etc., which act through G-protein coupled receptors includes all kinds of chemical messengers: ions (calcium ions acting on the parathyroid and kidney chemosensor), amino acids (glutamate and—amino butyric acid—GABA), monoamines (catecholamines, acetylcholine, serotonin, etc.), lipid messengers (prostaglandins, thromboxane, anandamide, (endogenous cannabinoid), platelet activating factor, etc.), purines (adenosine and ATP), neuropeptides (tachykinins, neuropeptide Y, endogenous opioids, cholecystokin in, vasoactive intestinal polypeptide (VIP), plus many others), peptide hormones (angiotensin, bradykinin, glucagon, caldtonin, parathyroid hormone, etc.), chemokines (interleukin-B, RANTES, MIP-1alpha etc.), glycoprotein hormones (TSH, LH/FSH, choriongonadotropin, etc.), as well as proteases (thrombin).

G-protein coupled receptors share a common mechanism of action. Binding of an extracellular ligand leads to a conformational change in the receptor protein that allows it to make contact with a G-protein which are located on the cytoplasmic side of the plasma membrane and which relays the extracellular signal from the activated G-protein coupled receptors to downstream intracellular effector proteins.

The G protein is a heterotrimer consisting of α, β and γ subunits. Currently, heterotrimeric G proteins are classified according to the nature of the α subunit and are grouped into four families (Gαₛ, Gα_{q}, Gα_{i/o}, and Gα_{12/13}) based on structural and functional similarity (Simon et al., 1991).

G-protein coupled receptors are classified according to the G-proteins that they contact. GPCRs of the Gs class mediate stimulation of adenylate cyclase via activation of Gαs, thereby increasing the intracellular level of CAMP. GPCRs of the Gi class mediate the inhibition of adenylate cyclase via activation of Gαᵢ, leading to a decrease in the intracellular level of cAMP. GPCRs of the Gαq class mediate stimulation of various phospholipase Cβ isoforms via activation of Gαq and lead to hydrolysis of membrane-bound phosphatidylinositol 4,5-bisphosphate to give diacylglycerol and inositol 1,4,5-triphosphate. Inositol 1,4,5-triphosphate causes the rapid release of Ca²⁺ from intracellular stores.

Typically, a given GPCR only interacts with one Gα protein subunit family, and, therefore, are selective for a particular signal transduction pathway. The narrow specificity of the G-protein coupled receptors is a hindrance to the identification of modulators of the G-protein coupled receptor-dependent signal transduction pathways. Moreover, a suitable signal, which can be utilized in a screening assay with high sample throughput, is currently obtained only from those signal transduction pathways in which, for example, G-protein activation leads to an increase in the intracellular Ca²⁺ level.

Thus, it would be very useful if a G-protein were available that is responsive to other G-protein coupled receptor classes, and which could also give a strong signal in the cell.

Extensive investigation of receptor peptides and chimeras has shown that the third intracellular loop sequence determines Gα coupling selectivity more often than does the second intracellular loop. Likewise, the first intracellular loop rarely determines specificity (Strader et al., 1994; Strader et al., 1995). On the level of the G protein α subunit, several different regions have been implicated in recognition of seven TM GPCRs and thus determine the specificity of receptor-G protein coupling; the extreme C-terminus, the extreme N-terminus, a region between the α4 and α5 helices, the α3/β5 loop, and a region within the loop linking the N-terminal α-helix to the β1-strand of the ras-like domain (Blahos et al). Current models of receptor-C protein interaction based on available crystal structures of G protein α subunits in active and inactive conformations predict these four regions to be in direct contact with the GPCR and determine the specificity of receptor-G protein coupling. Until now, no region within the G protein α subunit is known that influences the fidelity of GPCR-G protein interaction from a distance.

### DESCRIPTION OF THE INVENTION

The present inventors have found that novel mutants of the q-type Gα proteins, wherein the mutations are performed in regions that are not in direct contact with the G-protein coupled receptor, may be activated by non q-type G-protein coupled receptors. Furthermore, the inventors have shown that the novel mutants are superior compared to the wild type and other known q-type G protein mutants with respect to stimulation of the phospholipase Cβ-IP₃-Ca²⁺ signalling pathway.

X-ray crystallographic studies have revealed that Gα subunits consist of a GTPase domain and a helical domain, which are connected by two highly conserved linker regions (Sunhara et al., 1997; Noel et al., 1993; Coleman et al., 1994). According to x-ray structures, the guanine-nucleotide binding site lies in a cleft between these two domains and about 30 Å away from the cytoplasmic surface of the plasma membrane. Mutational alterations of the linker I and/or linker II regions have not been performed until now, nor has these regions been implicated in functions other than keeping both Gα domains in the proper distance from each other. The linker I region lies in close proximity to two of the three “switch regions' in the Gα structure that undergo conformational rearrangement during nucleotide exchange. The linker II region is identical to the switch I region known to undergo severe conformational change upon Gα activation. Linker II connects the α-helical domain of Gα to its the ras-like domain. Switch I (-linker II) is an important part of the lip for a potential exit route for GDP from the nucleotide binding pocket.

Molecular cloning of cDNAs has revealed that there are five subtypes of α-subunits belonging to the Gαq family: Gα_{q}, Gα₁₁, Gα₁₄, Gα₁₅ and Gα₁₆ (Simon et al., 1991). All Gα_{q} family members share common functional properties: they regulate the activity of phospholipase Cβ isoforms upon activation by G-protein coupled receptors. This leads to an increase in the intracellular level of inositolphosphates (IPs) in a pertussis-toxin insensitive manner (Lee et al., 1992).

Before going into details with the individual steps of the method of the invention, in the following is given a list of specific terms used in the present text.

Definitions

By the term “ligand” is intended to mean a substance that either inhibits or stimulates the activity of a receptor and/or that competes for the receptor in a binding assay.

An “agonist” is defined as a ligand increasing the functional activity of a receptor. A “partial agonist” is an agonist, which, no matter how high a concentration is applied, is unable to produce maximal activation of the receptors.

An “inverse agonist” (also termed “negative antagonist”) is defined as a ligand decreasing the basal functional activity of a receptor. Inverse agonism is a property of the ligand alone on the receptor. A “partial inverse agonists” only decreases the basal activity of the receptor to a certain level and not fully.

An “antagonist” is defined as a ligand decreasing the functional activity of a receptor by inhibiting the action of an agonist. In other words antagonism is a property of the ligand measured in the presence of a compound with higher signalling efficacy—i.e. usually a full agonist.

By the term “allosteric enhancer” is intended to mean a compound, which binds to a 10 second site on the receptor or a neighboring receptor, thereby enhancing response the ligand is evoking.

By the term “allosteric inhibitor” is intended to mean a compound, which binds to a second site on a receptor or a neighboring receptor, thereby preventing the ligand from evoking a response.

By the term “conserved amino acid(s)” is intended to mean a fragment of an amino acid sequence of a peptide, a polypeptide or a protein such as, e.g. the sequence of linker I from a q-type Gα protein, which compared to a fragment of an another amino acid sequence has at least about 40% identity, such as, e.g., at least about 45% or at least about 50% identity.

By the term “conservatively substituted amino acid(s)” is intended to denote that one or more amino acids is replaced by another, biologically similar residue. Examples include substitution of amino acid residues with similar characteristics, e.g. small amino acids, acidic amino acids, polar amino acids, basic amino acids, hydrophobic amino acids and aromatic amino acids. Non-limiting examples of conservative amino acid substitutions suitable for use in the present invention include the following

| Original residue | Conservative substitution |
|---|---|
| Ala | Gly, Val |
| Arg | Lys |
| Asn | Gln, His, Thr |
| Asp | Glu |
| Gln | Asn, His |
| Glu | Asp |
| His | Asn, Gln |
| Ile | Leu, Val |
| Leu | Ile, Val |
| Lys | Arg |
| Met | Leu, Ile |
| Phe | Tyr, Trp, His |
| Ser | Thr, Asn |
| Thr | Ser, Asn |
| Trp | Tyr, Phe, His |
| Tyr | Trp, Phe, His |
| Val | Ile, Leu, Ala |

In the present context the term “sequence identity of at least about 80%” is intended to indicate that the amino acid sequence of the peptide on average may include up to 2 amino acid alterations per each 10 amino acid residues of the specific amino acid sequence. In other words, to obtain a peptide having an amino acid sequence of at least 80% identity to a specific sequence, up to 20% of the amino acid residues in the subject sequence may be mutated, inserted, deleted, or substituted with another amino add residues. In analogous manner a sequence identity of at least about 50% means that the amino acid sequence of the peptide on average may include up to 5 amino acid alterations per each 10 amino acid residues of the specific amino acid sequence.

The “Linker I region” is a one out of 2 major regions connecting the α-helical domain of Gα to its ras-like domain. The linker I contains 5 amino acids.

The Linker 11 region” is the other out of 2 major regions within Gα that connects the α-helical domain to its ras-like domain. The linker II contains 11 amino acids.

The term “modulator” refers to a compound, which modulates a receptor, including agonists, antagonists, allosteric modulators and the like. Typically, the modulator binds to the receptor (i.e. it acts as a ligand for the GPCR). GPCR modulators thus refer to agents that modulate (e.g. stimulate or inhibit) the activity of G-protein coupled receptors.

As shown in the Examples herein, the present inventors have found that by introducing mutations in the linker I region of q-type Gα proteins, the proteins lose their ability to discriminate between GPCR functional classes and links Gαᵢ and Gαₛ-coupled receptors to the phospholipase Cβ-IP₃-Ca²⁺ signalling pathway. It has not been shown earlier that fidelity of receptor G protein interaction can be governed by mutations in regions other than those at the direct receptor G protein interface. The novel mutants may constitute a new tool that can be used in assays when promiscuous coupling is desired such as, e.g., searching for modulators of orphan G protein coupled receptors. Although the mutations are made in the linker I region, it is contemplated that mutations in the linker II region may lead to similar results. Accordingly, the present invention relates to a q-type G protein, wherein one or more mutations are made in the linker I and/or linker II region.

Thus, the present invention relates to a q-type G protein comprising one or more mutations in the linker I and/or linker II region, the one or more mutations making the q-type G protein responsive to one or more none type G protein coupled receptors. At the same time, the q-type G proteins are of course still responsive to G protein coupled receptors of type q. In a specific embodiment the one or more mutations make the q-type G protein responsive to at least two functional classes of non-q type G protein coupled receptors (e.g. Gi type and Gs type).

The non-q type G protein coupled receptors, i.e. receptors that under normal conditions do not activate Gα proteins of type q, may be Gi type receptors. Below are given some specific examples of Gi type receptors, but the invention of course relates to mutated G proteins that are capable of interacting with any G protein coupled receptors of type i.

Examples of Gi type receptors are receptors of the biogenic amine family, such as, e.g., muscarinic M2 and dopamine D2 receptors, peptide receptors, such as, e.g. somatostatin SSTR1 and kappa opioid KOR receptors and chemokine receptors, such as, e.g., CXCR1 and CXCR2 receptors. The invention is not limited to the specifically mentioned receptors but includes any Gi-linked GPCR.

Another functional class of receptors, which may interact with the mutated q-type G protein are Gs type receptors. Examples of Gs type receptors are the biogenic amine receptors, such as, e.g., dopamine D1 and adrenergic beta 2 receptor, family B receptors, such as, e.g., glucagon like peptide (GLP1) receptor and glucose dependent insulinotropic peptide (GIP) receptor. Again, the invention should of course not be limited to the specific receptors mentioned but include any G protein coupled receptors of type s.

The mutations performed in the linker I and/or II region of the q-type Gα proteins may be deletions, substitutions and/or insertions. The present invention includes any mutation in the linker I and/or linker II region, i.e. any of the amino acids of the linker I and/or linker 11 region may be substituted by any other amino acids, may be deleted and/or there may be inserted an arbitrary number of amino acids at arbitrary positions in the linker I and/or linker II sequence.

In one aspect the invention relates to a type q Gα protein, wherein the one or more mutations are substitutions of at least one amino acid in the linker I and/or in the linker 11 region, such as, e.g. at least two amino acids, at least three amino acids, at least four amino acids, at least five amino acids, at least six amino acid, at least seven amino acids or at least eight amino acids in respect of linker I and at least two amino acids, at least three amino acids, at least four amino acids, at least five amino acids, at least six amino acid, at least seven amino acids, at least eight amino acids, at least nine amino acids, at least ten amino acids or at least eleven amino acids in respect of linker II. There may be introduced substitutions in both the linker I and the linker II region in the same Gα protein, and the number of amino acids substituted in the two regions does not have to be the same.

As shown in FIG. 1, the linker I region comprises several conserved amino aids. The linker II region is also very well conserved. The sequence of linker II is about at least 80% identical in all Gα protein subunits. An example of the sequence of linker II may be SRVKTTGIIET. The present invention relates to q-type Gα proteins wherein one or more of the conserved amino acids in the linker I and/or linker II region may be substituted by any amino acid. Specifically, glycine 66 has been replaced with asparagine (N), valine (V), lysine (K), and aspartate (D).

A specific example of a mutation according to the invention is the introduction of a point mutation of a highly conserved glycine (glycine 66 in Gα_{q}) that upon mutation looses its ability to discriminate between GPCR functional classes and links Gαᵢ and Gαₛ-coupled receptors to the phospholipase Cβ-IP₃Ca²⁺ signalling pathway.

Accordingly, the present invention relates to a q-type G protein, which is a Gα_{qG66X} mutant, wherein X represents any amino acid. As shown below, the specific position of the conserved glycine will of course depend on the species and type of q protein used, i.e. the invention also includes mutants wherein the conserved glycine has another position than 66.

In a specific embodiment of the invention the glycine at position 66 may be exchanged with aspartate, valine, asparagines, or lysine giving a Gα_{qG66D/V/N/K} mutant. The sequences of the mutants are shown in SEQ ID No. 1.

As mentioned above at present five subtypes of the Gα protein are known. Accordingly, the invention also relates to Gα₁₁ and, more specific, to a Gα_{11G66X} mutant and a Gα_{11G66D} mutant. The sequence of the Gα_{11G66D} mutant is shown in SEQ ID No. 2.

The invention also relates to Gα₁₄ and more specific, to a Gα_{14G62X} mutant and a Gα_{14G62D} mutant. The sequence of the Gα_{14G62D} mutant is shown in SEQ ID No. 3.

The invention further relates to a q-type G protein, which is Gα₁₅ and to a Gα_{15G69X} and Gα_{15G69D} mutant. The sequence of the Gα_{15G69D} mutant is shown in SEQ ID No. 4.

The invention also relates to a q-type G protein, which is Gα₁₈ and furthermore to a Gα_{16D69X} and a Gα_{16G69D} mutant. The sequence of the Gα_{16G69D} mutant is shown in SEQ ID No 5.

Compared to the other members of the Gα_{q} family, Gα₁₅ and Gα₁₆ are unique in that they allow a variety of different receptors to productively stimulate PLCβ and intracellular IPs (Offermanns & Simon, 1995). The promiscuity of Gα₁₅ and Gα₁₆ make them ideal tools to be used in high throughput screening systems of G protein coupled receptors using intracellular calcium mobilization as readout. Intracellular calcium increases upon stimulation of GPCRs can be measured in a high throughput fashion using the Novo Star (BMG technologies) or the FLIPR (Molecular Devices Corp).

However, the fact that GPCRs with known ligands like the dopamine D3, chemokine CCR1- and CCR2-, angiotensin2 subtype 2-, somatostatin SST1-, adrenergic α1A-, α1C- and α1D-, melatonin 1c, muscarinic M1 and neurokinin B2-receptor do not or only poorly couple to Gα₁₆ (Mody et al., 2000; Milligan, G et al., 1996; Marchese et al., 1999; Lee et al., 1998, Kostenis, 2001) may suggest that Gα₁₆ does not represent a true universal tool for switching receptor signalling to intracellular calcium mobilization. Likewise, receptors such as the kappa opioid receptor or the dopamine receptor respond with very poor increases of IPs in the presence of coexpressed Gα₁₆. In Example 2 is shown that both the CXCR1 and D2 receptor mobilize no or very little calcium when stimulated with agonist ligand. In the presence of coexpressed Gα₁₆, both receptors gain the ability to stimulate calcium mobilization. However, upon coexpression of the receptors with Gα_{16D69D}, functional responses are significantly increased as compared to calcium signals upon coexpression with Gα_{16wt}. Thus, it is shown that Gα_{18wt} is superior to Gα_{16wt} in channeling the dopamine D2 and CXCR1 receptor to intracellular calcium mobilization.

It has earlier been described that N- and C-terminal alterations of q-type Gα proteins may be used to change coupling specificity of a given Gα subunit. One attempt has been to exchange some of the amino acids from the q-type G protein with the corresponding amino acids from an Gᵢ- or Gₛ-type protein, thereby conferring onto Gᵢ and Gₛ-linked receptors the ability to stimulate the G_{q} pathway. This mutation may be combined with the novel mutations according to the present invention. Such mutated chimeric proteins are envisaged to have improved properties compared to the non-mutated chimeric proteins.

Thus, the invention relates to a q-type G protein, which is further altered by replacing one or more amino acids at the C-terminus of the protein with the corresponding amino acids from an i-type G-protein. In a specific embodiment the last five amino acids at the G terminus are replaced with the corresponding amino acids from an i-type G-protein. A specific example is the Gα_{qG68Xi5} mutant such as the Gα_{qG66Di5} mutant. The mutant may have a sequence as shown in SEQ ID No. 6.

The invention also relates to a q-type G protein, which is further altered by replacing one or more amino acids at the C-terminus of the protein with the corresponding amino acids from a s-type G-protein. In a specific embodiment the last five amino acids at the C-terminus are replaced with the corresponding amino acids from a s-type G-protein. A specific example is the Gα_{qG66Xa5} mutant such as the Gα_{qG88Da5} mutant. The mutant may have a sequence as shown in SEQ ID No. 7.

Surprisingly, as shown in the Examples the inventors have found that coexpression of a non-Gq-selective receptor with the Gα_{qG66Di5} or the Gα_{gG66Ds5} mutant induced a robust response exceeding the response observed upon coexpression with the C-terminal or the Gα_{wG66D} mutant alone, i.e. the combination of the G66D mutation with the C-terminal alteration gave rise to a mutant which is superior to the individual mutants with respect to stimulation of PLCβ.

The invention relates to an isolated nucleic acid molecule comprising a polynucleotide encoding a q-type G protein according to the invention. Furthermore, the invention relates to a recombinant DNA expression vector comprising such a nucleic acid molecule, and to a host cell transformed with the vector. The recombinant DNA expression vectors used may be any suitable inducible or non-inducible expression vectors, such as, e.g. mammalian, avian, yeast or insect cell expression vector. The host cells may be any suitable host cells, such as, e.g. mammalian cells, such as, e.g. COS7, CHO, HEK293 cells or yeast cells. As mentioned above the novel mutants may be used in a method for identifying modulators of G-protein coupled receptors.

Accordingly, the invention relates to a method for identifying modulators of a G-protein coupled receptor in a sample, comprising the steps of
a) contacting a cell comprising one or more O-protein coupled receptors and one or more q-type G proteins according to the invention with the sample,
b) detecting any q-type G protein response.

Suitable conditions for allowing binding of modulators to the receptor may be physiological conditions wherein the pH is maintained between 6 and 8, and the temperature is between about 20°-40° C.

The GPCR may be a non-Gq type receptor, such as a Gi-type receptor or a Gs-type receptor. Specific examples of receptors are mentioned above, but the application intends to include all nonGq type receptors. The G-protein coupled receptor may also be an orphan receptor, i.e. a receptor for which the binding partner is unknown.

As described above the q-type G-protein is coupled to the phospholipase Cβ-IP₃-Ca²⁺ signalling pathway. Thus, in a method according to the invention the detection may comprise measuring phospholipase C, hydrolysis of phosphatidylinositols and/or calcium mobilization.

In a specific embodiment the detecting comprises measuring of calcium mobilization. One way of detecting calcium mobilization is with calcium sensitive dyes. Cells are incubated for about 60-120 minutes with a calcium sensitive dye such as Fluo3 or Fluo4 (Molecular devices corp). These calcium sensitive dyes emit light at different wave lengths when bound and unbound, respectively to calcium. Upon release of intracellular calcium induced by stimulation of a 7TM OPCR, calcium is bound by the dye and the dye emits light at a certain wavelength that can be measured with instruments such as the Novostar or the FLIPR (Fluorometric imaging plate reader).

Calcium mobilization is amenable to high-throughput screening using robotic assays. Thus, the invention also relates to a high-throughput screening method comprising a method according to the invention.

The modulator, which may be identified by a method according to the invention, may be an antagonist, a partial antagonist, an inverse antagonist, an agonist, a partial agonist, an inverse agonist, an allosteric enhancer or an allosteric inhibitor.

The method according to the invention may be used for identifying one or more lead compounds for use in drug discovery methods, for identifying one or more lead compounds for use in target validation methods, for identifying one or more lead compounds for use in a chemogenomic model and for identifying a compound for use in medicine and/or diagnosis.

The invention further relates to a compound identifiable by a method according to the invention. The compound may be used in a drug discovery method, in a target validation method, in a chemogenomic model and/or in medicine.

The invention also relates to a pharmaceutical composition comprising a compound for use in medicine and/or diagnosis identifiable by a method according to the invention together with a pharmaceutically acceptable excipient. A person skilled in the art will know how to formulate pharmaceutical composition optionally with guidance from handbook like e.g. Remington's Pharmaceutical Science.

Other Aspects of the Invention

Other aspects of the invention appear from the appended claims. The details and particulars described above and relating to a mutated q-type G protein according to the invention apply mutatis mutandis to the other aspects of the invention.

### FIGURE LEGENDS

FIG. 1 shows a sequence alignment and secondary structure features of the extreme N-termini, the N-terminal α-helices and the linker I region of various G protein α subunits. Gaps were introduced for optimum sequence alignment. Gα_{qG66X} denotes mutant Gα_{q} constructs in which glycine has been mutated to aspartate, valine, asparagine, or lysine in the linker I region. Gen bank accession numbers for the Gα proteins listed from top to bottom are Gαq, Gα11, Gαi1 (P04898), Gas (P04896), Gat (P04695).

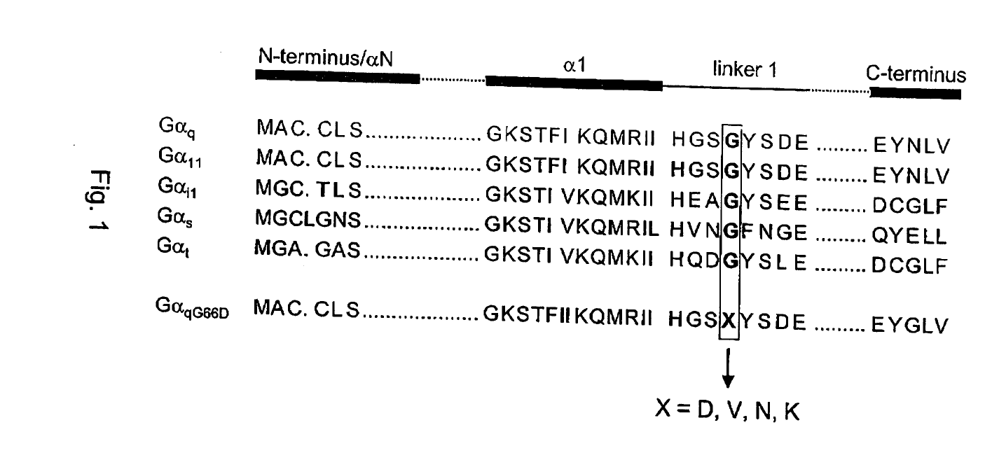

FIG. 2 shows the functional interaction of selected Gi-coupled receptors with Gα_{q}wt and Gα_{qG66D}. COS7 cells were cotransfected with expression plasmids coding for the muscarinic M2, dopamine D2, chemokine CCR5, and sphingosin-1-phosphate S1P5 (formerly referred to as edg8) receptor and the indicated G proteins or pCDNA1-vector DNA as a control. Transfected cells were incubated for 30 min (at 37° C.) in the absence or presence of various concentrations of the appropriate agonist ligands. Basal IP₃ levels (no ligand added) were similar for vector and Gα subunit transfected cells (not shown). The resulting increases in intracellular IP₃ levels (fold stimulation above basal) were determined as described under “Examples.” Data are given as means±S.E. of three to five independent experiments, each carried out in triplicate.

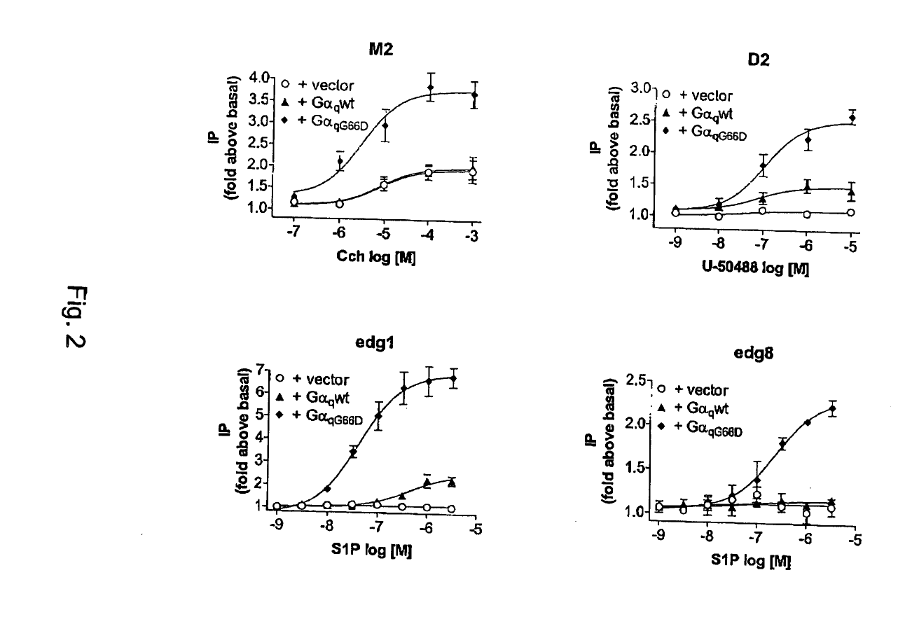

FIG. 3 shows the functional interaction of selected Gi-coupled chemokine receptors with Gα_{q}wt and Gα_{qG88D}. COS7 cells were cotransfected with expression plasmids coding for the chemokine CCR5 receptor and the indicated G proteins or pCDNA1-vector DNA as a control. Transfected cells were incubated for 30 min (at 37° C.) in the absence or presence of various concentrations of the appropriate agonist ligands. Basal IP₃ levels (no ligand added) were similar for vector and Gα subunit transfected cells (not shown). The resulting increases in intracellular IP₃ levels (fold stimulation above basal) were determined as described under “Examples.” Data are given as means±S.E. of three to five independent experiments, each carried out in triplicate.

FIG. 4 shows the functional interaction of selected Gs-coupled receptors with Gα_{q}wt and Gα_{qG66D} COS7 cells were cotransfected with expression plasmids coding for the glucose-dependent insulinotropic peptide (GIP) receptor and the glucagons like peptid1 (GLP1) receptor and the indicated G proteins or pCDNA1-vector DNA as a control. Transfected cells were incubated for 30 min (at 37° C.) in the absence or presence of various concentrations of the appropriate agonist ligands. Basal IP₃ levels (no ligand added) were similar for vector and Gα subunit transfected cells (not shown) The resulting increases in intracellular IP₃ levels (fold stimulation above basal) were determined as described under “Examples”. Data are given as means±S.E. of three independent experiments, each carried out in triplicate.

FIG. 5 shows the functional interaction of selected Gi- and Gs-coupled receptors with Gα_{q}wt and the Gα_{qG66X(X=D,V,N,K)} mutants. COS7 cells were cotransfected with expression plasmids coding for the glucose-dependent insulinotropic peptide receptor (GIP), glucagon like peptide 1 (GLP1) receptor, prostaglandin D2 receptor (DP), chemokine CCR5 receptor, adrenergic α2A receptor, and chemokine ChemR23 receptor and the indicated G proteins or pCDNA1.1-vector DNA as a control. Transfected cells were incubated for 30 min (at 37° C.) in the absence or presence of various concentrations of the appropriate agonist ligands. Basal IP₃ levels (no ligand added) were similar for vector and Gα subunit transfected cells (not shown). The resulting increases in intracellular IP₃ levels (fold simulation above basal) were determined as described under “Examples.” Data show one out of three representative experiments, each carried out in duplicate.

FIG. 6 shows a functional characterization of the Gα_{q}wt and Gα_{qG66D} proteins. Basal IP₃ formation was determined in COS7 cells transiently transfected with vector DNA, Gα_{q}wt, Gα_{qG66D} and the constitutively active Gα_{15Q212L} mutant as described in detail in the Examples. Data represent mean values±SEM of 4 independent experiments performed in duplicate.

FIG. 7 illustrates AIF4⁻-mediated IP₃ production of Gα_{q}wt and Gα_{qG66D} proteins. AIF4⁻ stimulated IP₃ production was determined in COS7 cells transiently transfected with vector DNA as a control, Gα_{q}wt, or Gα_{qG66D} as described in detail in Examples. Data represent mean values±SEM of 4 independent experiments performed in duplicate.

FIG. 8 shows the mapping of residues important for receptor-G protein coupling specificity onto a receptor-G protein model. Positioning of a heterotrimeric G protein (Gαₜ-β-γ) relative to a 7TM receptor is based on a model proposed by Iiri et al. (1998). Regions known to determine specificity of receptor-C protein interaction are the extreme C-terminus, the α4-α6 loop and the small loop connecting the N-terminal α-helix with the β1 strand of the ras-like domain. The guanine nucleotide GDP is buried between the helical and the GTPase-domain of the Gα subunit. The 2 linker regions connect the helical and the GTPase-domain of Ga. The circle within linker I indicates the location of the G66D mutation imparting promiscuity onto Gα_{q}. The PDB identifiers for the receptor, Gα and βγ are 1F88, 1TBG and 1BH2 respectively

FIG. 9 shows an alignment of the N- and C-terminal amino acid sequences of selected mutant and wild type G protein α subunits. Gaps were introduced for optimum sequence alignment. Gα_{qi5} and Gα_{qa5} denote mutant Gα_{q} constructs in which the C-terminal five amino acids are replaced with the corresponding Gαᵢ(Gα_{qi5}) or Gαₛ (Gα_{qs5}) sequence, respectively. The boxed glycine residue in the linker I region is highly conserved between all known Gα subunits, even from distantly related organisms. Gα_{qG66D} is a mutant α subunit with a glycine to aspartate mutation in the linker I region. This mutant has previously been shown to impart promiscuity onto Gα_{q} in that it allows Gi- and Gs-linked receptors to activate the PLCβ pathway. Gα_{qG66Xi5/s5} are mutant Gα subunits combining a C-terminal sequence switch and the G86X (X=D,V,N,K) mutation in linker I. The single letter amino acid code is used.

FIG. 10 shows an expression analysis of wild type and mutant Gα_{q} proteins in COS7 cells. COS7 cells were transfected at 50% confluency in 100 mm dishes with pCDNA1 vector DNA or the indicated G protein α subunits (8 μg plasmid DNA/dish) using the Fugene transfection reagent according to the manufacturer's instructions. 48 h after transfection membranes were prepared and analyzed (2040 μg each) by SDS-polyacrylamide gel electrophoresis (13%) and Western blotting using the 12CA5-peroxidase linked monoclonal antibody as described under “Examples”. Similar results were obtained in two additional experiments with different batches of membranes.

FIG. 11 shows the functional interaction of selected Gi-coupled receptors with Gα_{q}wt, Gα_{qG66D}, Gα_{qi5}, and Gα_{QG66Di5}. CO₅₇ cells coexpressing pCDNA1 vector DNA or the indicated Gα subunits and different Gi-linked receptors (chemokine CXCR2, chemokine CCR5, somatostatin receptor subtype 1 (SSTR1), kappa opioid receptor (KOR), GPR7, HM74a, neuropeptide Y receptor subtype 4 (NPY4), muscarinic M2. P2Y12) were incubated for 45 min (37° C.) in the absence or presence of increasing concentrations of the appropriate agonist ligands. The resulting increases in intracellular IP levels were determined as described under “Examples”. Data are means±SE of three to five experiments each performed in duplicate.

FIG. 12 shows the functional interaction of selected Gs-coupled receptors with Gα_{q}wt, Gα_{qG66D}, Gα_{qs5}, and Gα_{qG66Da5}. COS7 cells coexpressing pCDNA1.1 vector DNA or the indicated Gα subunits and the glucagone like peptide 1 (GLP1), the glucose-dependent insulinotropic peptide (GIP) receptor, the prostaglandin D2 DP receptor, or the adrenergic P2 receptor were incubated for 45 min (37° C.) in the absence or presence of increasing concentrations of the appropriate agonist ligands. The resulting increases in intracellular IP levels were determined as described under “Examples”. Data are means±SD of two independent experiments each performed in duplicate.

FIG. 13 shows agonist dose response curves for the dopamine 2 receptor selective agonist (−)quinpirole and the CXCR1 agonist interleukin 8 in COS7 cells transiently cotransfected with the indicated receptor and G protein expression plasmids. 24 h after transfection, COS7 cells were split into 96 well plates at a density of ˜30,000 cells per well. 18-24 h later cells were loaded with a calcium sensitive dye (calcium assay kit from Molecular devices) for 1 h (37 C, 5% CO₂) and then for 1 h at room temperature. Ligands were transferred to the cell plate from the sample plate using the Novostar's integrated pipette. To obtain the specific fluorescence response, baseline fluorescence counts before addition of ligands were subtracted from the peak of the calcium response Specific responses were used to plot the concentration response curve. Gα_{16G69D} is superior to Gα₁₆ in channelling the dopamine D2 and CXCR1 receptor to intracellular calcium mobilization.

FIG. 14 shows the functional interaction of selected Gs- and Gi-coupled receptors with Gα_{qs5/i5/z5}, Gα_{qG66DXa5/z5/i5 (X=D,N,V,K)}. COS7 cells coexpressing pCDNA1.1 vector DNA or the indicated Gα subunits and the glucagone like peptide 1 (GLP1) receptor, the prostaglandin D2 DP receptor, the neuropeptide Y NPY4 receptor, or the chemokine ChemR23 receptor were incubated for 45 min (37° C.) in the absence or presence of increasing concentrations of the appropriate agonist ligands. The resulting increases in intracellular IP levels were determined as described under “Examples”. Data show results of a representative experiment each performed in duplicate.

### EXAMPLES

### Example 1

Gα_{qG66X(X=D,N,V,K)}

Materials and Methods

DNA Construction

Murine wild type Gα_{q} was cloned from mouse brain by RT-PCR and inserted into the BamMI-NsiI-sites of the pcDNA1.1 expression plasmid. Both, wild type Gα_{q} and Gα_{qG66X} contained an internal hemagglutinin epitope tag DVPDYA (Wedegaertner et al., 1993) replacing Gα_{q}wt residues 125-130. Gα_{qG66X} mutants were prepared with the Stratagene quick change mutagenesis kit according to the manufacturer's instructions. The correctness of all PCR-derived sequences was verified by sequencing in both directions.

Cell Culture and Transfections

COS7 cells were grown in Dulbecco's modified Eagles medium supplemented with 10% (vol/vol) fetal calf serum, Penicillin-Streptomycin (10000 IU/ml-10000 μg/ml) and 2 mM L-Glutamin in a humidified 5% CO₂ incubator. For transfections, 1×10⁶ cells were seeded into 100-mm dishes. About 24 h later, cells were cotransfected with the indicated G protein and receptor constructs (1:1 ratio) by using the Fugene transfection reagent as per manufacturer's instructions.

PI-Hydrolysis Assays

18-24 h after transfections cells were split into 35 mm dishes in culture medium supplemented with 3 μCi/ml [3H]myo-inositol (20 Ci/mmol: Amersham). After a 24 h labeling period cells were incubated during 20 min at room temperature with 2 ml of HANK's balanced salt solution containing 20 mM HEPES and 10 mM LiCl. Cells were then stimulated in the same buffer with the appropriate agonist ligands for 45 min (37° C.), and increases in intracellular inositolphosphate determined by anion exchange chromatography as described (Kostenis et al., 1997)

Receptor Ligands

Rantes (recombinant, human) and Somatostatin 14 were from Bachem Biochemica GmbH, (−)-quinpirole and U-50488 from Research Biochemicals Inc. (RBI). The remaining chemicals were purchased from Sigma. Chemerin9 was synthesized by UFpeptides (Ferrara, Italy).

Rosults

Role of G66D Mutation in GI-Coupled Receptor-Mediated Activation of Phospholipase Cβ.

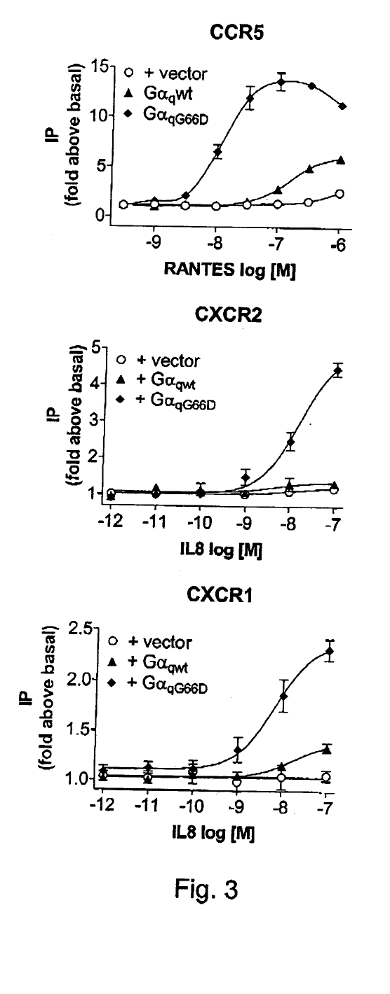

Transiently transfected COS7 cells were used cells to test whether the mutated α subunit differs from Gα_{q}-WT in its ability to be activated by Gi-coupled receptors in functional assays monitoring IP₁ production. To this end, COS7 cells were cotransfected with cDNAs encoding the Gi-linked muscarinic M2-, dopamine D2-, sphingosine-1-phosphate S1P1 (edg1)-, and sphingosine-1-phosphate S1P5 (edg8) receptor and vector DNA as a control or Gα_{q}-WT or Gα_{qG66D}, respectively. Vector-transfected cells or Gα subunit transfected cells did not respond to any concentrations of the agonists tested in this study (data not shown). Likewise, cotransfection of the various Gi-linked receptors upon cotransfection with Gα_{q}-WT did not (D2, S1P1, S1P5) or only moderately (M2) stimulate inositol phosphate hydrolysis. Interestingly, all tested receptors responded with a robust increase in inositolphosphates upon cotransfection with Gα_{qG66D}. Next the ability of three Gi-linked chemokine receptors (CCR5, CXCR1, CXCR2) to functionally interact with Gα_{q}wt and Gα_{qG66D} were examined. In agreement with their known coupling profile, these receptors were unable to stimulate Gα_{q}wt to an appreciable extent (FIG. 3). All three receptors, however, gained the ability to productively couple to Gα_{qG66D} (FIG. 3).

Role of G66D Mutation in Gs-Coupled Receptor-Mediated Activation of Phospholipase Cβ.

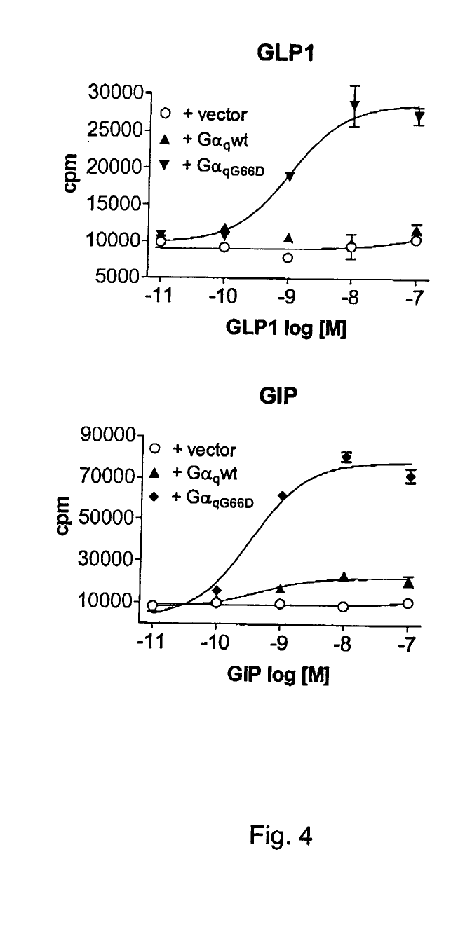

The ability of selected Gs-linked receptors to functionally interact with Gα_{q}-wt and Gα_{qG66D} were then determined. To this end, COS7 cells were cotransfected with cDNAs encoding the glucose-dependent insulinotropic peptide (GIP) receptor and the glucagon like peptide 1 (GLP1) receptor and vector DNA as a control or Gα_{q}-wt or Gα_{qG66D}, respectively. Cotransfection of the Gs-linked receptors with Gα_{q}-wt did not (GLP1 receptor) or only moderately (GIP receptor) stimulate inositol phosphate hydrolysis (FIG. 4). However, all tested receptors responded with a robust increase of inositolphosphates upon cotransfection with Gα_{qG66D} (FIG. 4). Thus, the G66D mutation appears to be a truly promiscuous Gα subunit allowing receptors otherwise not or only poorly coupled to PLCβ to mediate intracellular IP₁ accumulation.

Role of G66X(X=D,V,N,K) Mutation in Gi- and Gs-coupled receptor-mediated Activation of phospholipase Cβ.

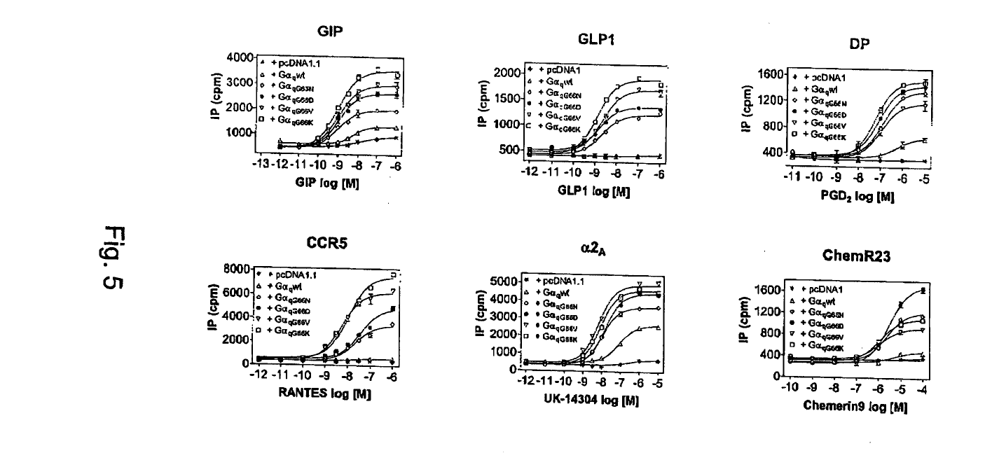

The ability of selected Gs- and Gi-linked receptors to functionally interact with Gα_{q}-wt and Gα_{qG66X} were then determined. To this end, COS7 cells were cotransfected with cDNAs encoding the glucose-dependent insulinotropic peptide (GIP) receptor, the glucagon like peptide 1 (GLP1) receptor, the prostaglandin D2 DP receptor, the chemokine CCR5 receptor, the adrenergic α2A receptor, or the chemokine ChemR23 receptor and vector DNA as a control or Gα_{q}-wt or the Gα_{qG66X} mutants, respectively. Cotransfection of the receptors with Gα_{q}-wt did not (GLP1 receptor, CCR5, ChemR23) or only moderately (GIP receptor, DP, α2A) stimulate inositol phosphate hydrolysis (FIG. 5). However, all tested receptors responded with a robust increase of inositolphosphates upon cotransfection with the Gα_{qG66X} mutants (FIG. 5). Thus, the G66X mutation appears to be a truly promiscuous Gα subunit allowing receptors otherwise not or only poorly coupled to PLCβ to mediate intracellular IP₁ accumulation.

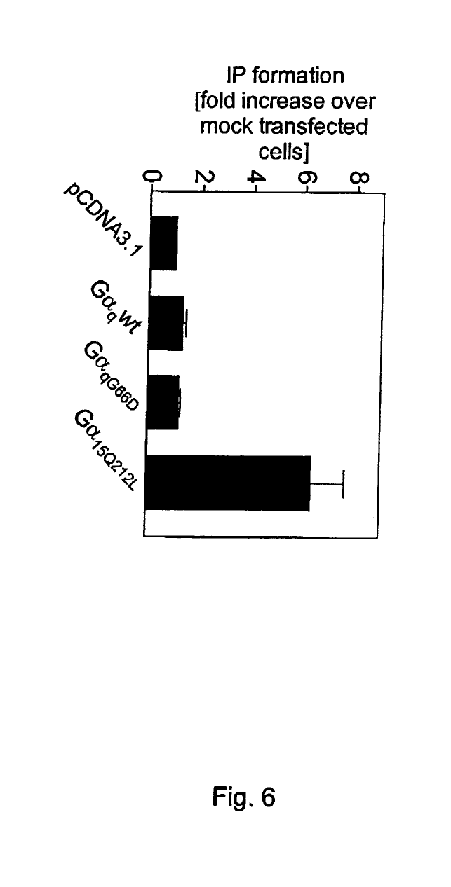

Gα_{qG66D} does not display a constitutively active phenotype: comparison of Gα_{qG66D} to GTPase-deficient mutants. To test whether the mutant Gα_{q} subunit differs from Gα_{q} wt in its ability to signal to phospholipase Cβ, plasmids encoding the respective cDNAs were transiently transfected into COS7 cells and IP₁ accumulation was measured. FIG. 6 shows the effect of the G66D mutation on PLCβ activation. No significant increase in the ability to activate PLCβ was caused by the mutation. For means of comparison, a GTPase defective Gα₁₅ mutant, referred to as the QL mutation, that replaces Glutamine 212 by Leucine and causes constitutive activation was included and induced a robust IP₁ increase as compared with the wild type Gα_{q} or Gα_{qG66D}.

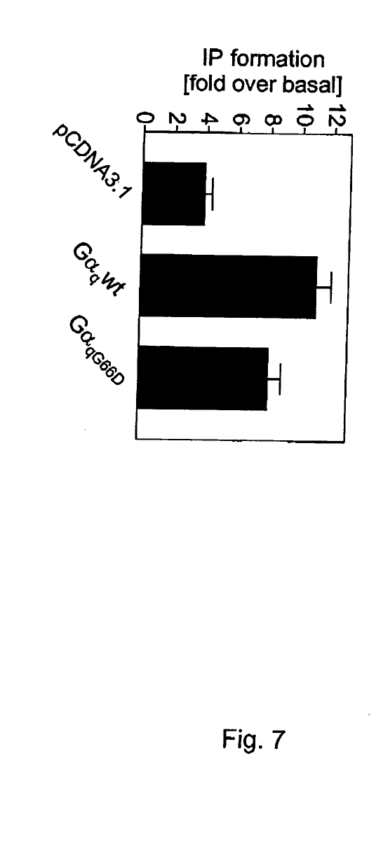

Gα_{G66D} exhibits impaired activation by AIF4⁻. It was tested whether the mutation interferes with the ability of fluoroaluminate (AIF4−) to activate the α subunit. Altered activation by AIF4− would indicate an interference of the mutation with the ability of Gα to bind the guanine nucleotide. AIF4− typically activates G protein α subunits by binding to the GDP-bound form of Ga. The resulting Gα-GDP-AIF4− complex assumes an active state conformation, which resembles that of Gα-GTP complex (Coleman et al., 1994). Both fluoroaluminate-activated and receptor-activated Gα subunits are capable of stimulating intracellular effector molecules. FIG. 7 depicts the effects of 300 μM AIF4⁻ on Gα subunit-mediated IP₁ production. Interestingly, the Gα_{qG66D} mutant is slightly impaired in its ability to mediate AIF4⁻-induced IP₁ production indicating a destabilization of the guanine nucleotide bound state.

Discussion

Several regions within the Gα protein sequence are known to be involved in the selectivity of its activation by cell surface 7 transmembrane receptors. By far the best characterized region is the extreme C-terminus of the Gα protein comprising the last 9 amino acids and it has been shown that mutational alteration of the C-terminal sequence comprising as few as one amino acid may be sufficient to change the coupling specificity of a given Gα subunit (Kostenis et al., 1997). Other regions determining coupling selectivity of Gα proteins comprise the extreme N-terminus (Kostenis et al., 1997), the region between α4 and α5 helices (a4-b6 loop) (Onrust et al., 1997; Mazzoni & Hamm, 1996; Bae et al., 1997; Hamm et al., 1988; Bockaert & Pin, 1999) and a region within the loop that links the N-terminal α-helix to the β1 strand of the ras like domain (Blahos et al., 2001). Evidence from mutagenesis and x-ray crystallographic studies as well as models of receptor G protein complexes based on these studies suggests all regions to be in intimate contact with the intracellular surface of the receptor protein. No region within Gα is known to date that controls specificity of receptor-G protein interaction from a region other than the postulated direct receptor-G protein interface.

G-protein alphα subunits consist of two domains: a Ras-like domain also referred to as GTPase domain, structurally homologous to monomeric G-proteins and so called due to its resemblance of the oncoprotein p21^{ras}, and a more divergent domain, unique to heterotrimeric G-proteins, referred to as helical domain. G-protein activation requires the exchange of bound GDP for GTP, and since the guanine nucleotide is buried in a deep cleft between both domains, it has been postulated that activation may involve a conformational change that will allow the opening of this cleft. Crucial for the opening process is the movement of the GTPase domain away from the helical domain and the linker regions are thought to act as hinges since they are relatively close together toward the phosphate end of the nucleotide (Noel, et al., 1993). Whereas the linker II region constitutes one of the three switch regions known to undergo severe conformational alteration upon Gα activation, nothing is known about linker I except for acting as a tether of the helical and ras-like domain, respectively (Coleman et al., 1994). There are, however, naturally occurring G protein variants that differ in the composition of the otherwise highly conserved linker I region the predominant splice variants of Gas (Gas short and Gas long) differ in the amino acid sequence of linker I in that linker I of Gas-long contains a 15-amino acid insert at position 72. Both splice variants have been shown to act differentially with G protein coupled receptors (Unson et al., 2000) and to exhibit differences in the kinetics of receptor-mediated nucleotide exchange or altered nucleotide affinity (Seifert et al., 1998). Whether the linker I region plays a role in specificity of receptor-G protein interaction has not been tested so far. We reported here about the identification of a Gα_{q} point mutation harboring a glycin to aspartate exchange in linker I. This mutant Gα_{qG66D} protein lost the ability to discriminate between functional GPCR classes and links Gi- and Gs-coupled receptors to the Gα_{q}-PLCb pathway. We could also show that promiscuous receptor coupling is not due to increased expression levels of the mutant G protein.

Furthermore, our mutant Ga_{qG66D} protein did not display a constitutively active phenotype. Constitutive activity of Gα proteins may arise from slow GTP hydrolysis (Masters et al., 1989; Landis et al., 1989; Graziano et al., 1989) or faster GDP release, which in turn allows it to more frequently associate with GTP and resume the active conformation (Iiri et al., 1994). Irrespective of the underlying mechanism, in both cases, increased activity reflects residence of the protein in its GTP-bound state for a larger proportion of time as compared with a wild type Gα protein. We can therefore infer from our results that Gα_{qG66D} does not display slower GTP hydrolysis given the absence of intrinsic PLCβ stimulation. The mutant does however display an alteration in its nucleotide binding pocket This notion is supported by the results of the AIF4⁻ assays as AIF4⁻0 mediated IP₁ production is significantly impaired in Gα_{qG66D} as compared with Gα_{q}wt. Given that AIF4⁻0 only binds to Gα-GDP, reduced Gα-GDP-AIF4⁻ complexes reflect reduced availability of Gα-GDP and thus a destabilized guanine nucleotide bound state.

The phenotype of our mutant is intriguing as it displays characteristics reminiscent of dominant negative Gα subunits (impaired AIF4⁻ activation, lack of constitutive activity and therefore stabilized empty pocket conformation) while at the same gaining the ability to channel Gi- and Gs-linked receptors to the PLCβ pathway. Indeed, when coexpressed with Gq-coupled receptors, Gα_{q66D} induces a rather modest decrease in IP₁ accumulation (˜20%) when stimulated by agonists for Gq-linked receptors (data not shown). Our mutant should therefore add to the repertoire of Gα proteins with stabilized a, state. The ae state can be stabilized by different mechanisms: impaired binding of GDP (Iiri et al., 1994 and this study), impaired binding of GYP or impaired GTP-induced conformational change (Lee et al., 1992; Iiri et al., 1997). Irrespective of the molecular mechanism underlying stabilization of the αₑ state, those mutants should represent precious proteins to understand the catalytic mechanism of the GTP-GDP exchange reaction of Gα subunits. Crystals have revealed three-dimensional structures of the GDP-bound αβγ complex and the GTP-bound Gα and uncomplexed βγ subunits (Noel et al., 1993; Coleman et al., 1994; Lambright et al., 1994; Mixon et al. 1995; Lambright et al., 1996: Sondek et al., 1996; Sunahara et al., 1997). To fully understand the catalytic mechanism of nucleotide exchange a Gα subunit with an empty nucleotide binding pocket would be highly desirable. Such a crystal remains elusive given that the empty pocket conformation, induced by association with a 7TM receptor, is highly labile and extremely shortlived (Iiri et al., 1994; Ross & Gilman, 1977: Ferguson et al., 1986). The mutant Gα, identified herein, could therefore constitute a precious tool for shedding more light onto the structural basis of the GDP/GTP exchange reaction.

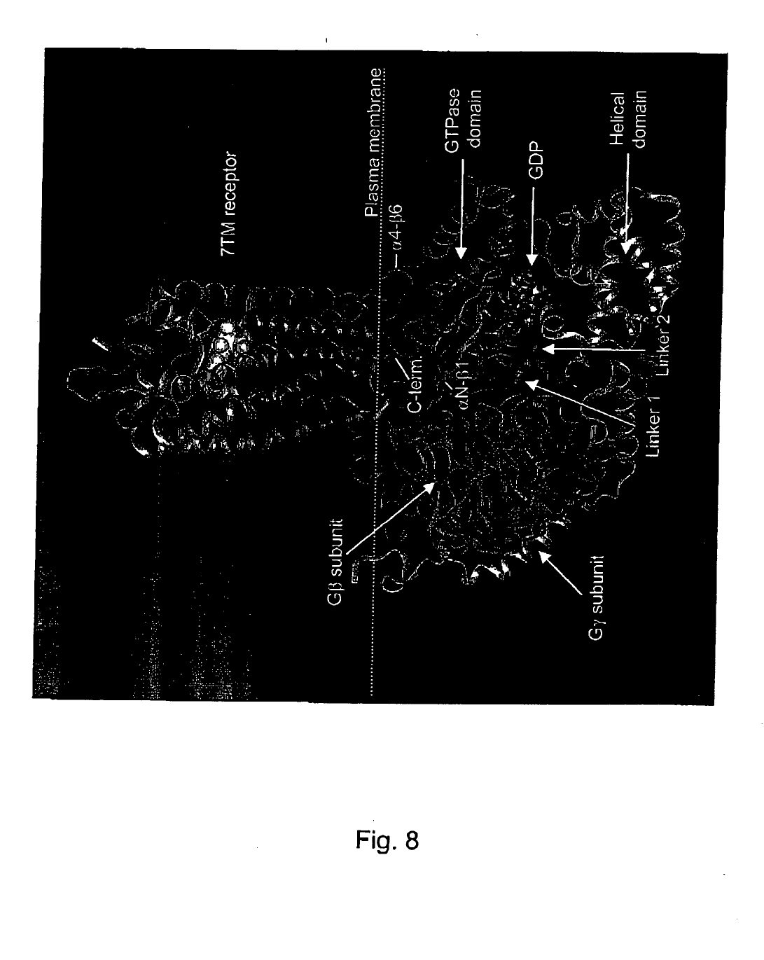

In conclusion, we have identified a new region within the Gα subunit that acts as a regulator of receptor-G protein coupling specificity most likely via acting from a distance of the proposed cytoplasmic receptor-C protein interface (FIG. 8). Although the precise molecular mechanism for inducing a specificity switch of Gα remains elusive, Gα_{qG66X(X=D,V,N,K)} constitute new tools for linking Gi- and Gs-coupled receptors to the PLCβ pathway and add to the repertoire of promiscuous G proteins. It is intriguing to note that the glycine residue is highly conserved in all Gq subunits even in sequences from distantly related organisms suggesting that Gq subunit functionality is similarly affected in other Gα proteins by the corresponding mutation.

### Example 2

Gα_{16G69D}

Materials and Methods

DNA Construction

Murine Wild type Gα₁₆ was cloned as described above. Gα_{18G69D} was prepared with the Stratagene quick change mutagenesis kit according to the manufacturers instructions. The correctness of all PCR-derived sequences was verified by sequencing in both directions.

Cell Culture and Transfections

COS7 cells were grown and transfected as described above.

Calcium Mobilization Assay

24 h after transfection, COS7 cells were split into 96 well plates at a density of −30.000 cells per well. 18-24 h later cells were loaded with a calcium sensitive dye (calcium assay kit from Molecular devices) for 1 h (37 C, 5% CO₂) and then for 1 h at room temperature. Ligands were transferred to the cell plate from the sample plate using the Novostar's integrated pipette. To obtain the specific fluorescence response, baseline fluorescence counts before addition of ligands were subtracted from the peak of the calcium response. Specific responses were used to plot the concentration response curve.

Results and Discussion

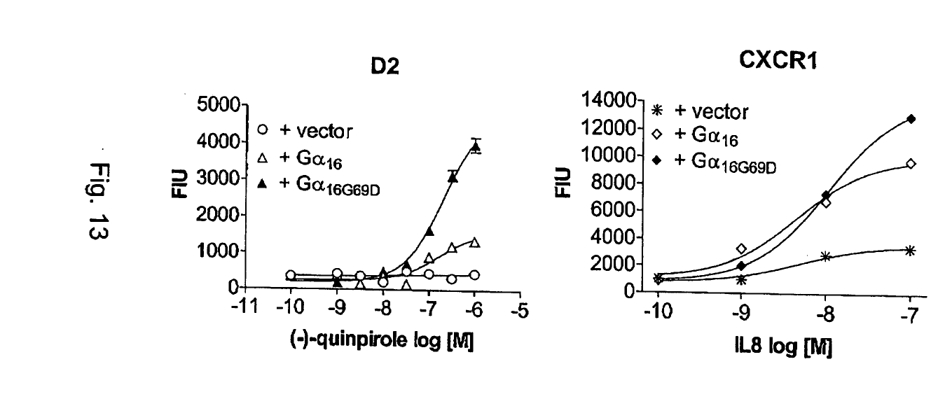

The present example intends to determine whether the glycine to aspartate mutation in the highly conserved linker I region of Gα₁₆ improves its ability to link poorly coupled receptors to the PLCβ pathway in an assay measuring intracellular calcium mobilization. To this end, the chemokine CXCR1 and the dopamine D2 receptor with vector DNA as a control or Gα₁₆ or Gα_{16G69D} were cotransfected and increases in intracellular calcium upon receptor stimulation with the appropriate agonist ligands were recorded. FIG. 13 shows that both the CXCR1 and D2 receptor mobilize no or very little calcium when stimulated with agonist ligand. In the presence of coexpressed Gα₁₆, both receptors gain the ability to stimulate calcium mobilization. However, upon coexpression of the receptors with Gα_{16G69D}, functional responses are significantly increased as compared to calcium signals upon coexpression with Gα_{16wt}. Thus, FIG. 13 shows that Gα_{16G69D} is superior to Gα_{16wt} in channeling the dopamine D2 and CXCR1 receptor to intracellular calcium mobilization.

These data clearly show that the glycine to aspartate mutation in the Gα_{q} subunit family renders them more susceptible towards signals of receptors not or poorly coupled to PLCβ. The mutant Gα_{16G69D} could constitute a precious tool in boosting Gα₁₆-mediated PLCβ stimulation or intracellular calcium mobilization in low and high throughput screening assays of 7TM GPCRs.

### Example 3

Gα_{qG66Xi5/s5/z5(X=D,N,V,K)}

Materials and Methods

DNA Construction

Murine wild type Gα_{q} was cloned from mouse brain by RT-PCR and inserted into the BamHI-NslI-sites of pcDNA1.1. All wild type and mutant Gα subunits contained an internal hemagglutinin epitope tag DVPDYA (Wedegaertner et al., 1993) replacing Gα_{q}wt residues 125-130. To create the C-terminally modified Gα_{qi5} subunit, in which the last five aa of wt Gα_{q} were replaced with the corresponding Gαᵢ sequence, a 175-bp BglII-NslI fragment was replaced, in a two piece ligation, with a synthetic DNA fragment, containing the desired codon changes. Gα_{qs5/z5} were prepared in an analogous fashion. Gα_{qG66x} mutants were prepared with the Stratagene quick change mutagenesis kit according to the manufacturer's instructions. To generate Gα_{qG66Xi5/s5/z5(X=D,V,N,K)}, a 175 bp BglII-NsiI fragment was removed from GαqG66X and replaced with the corresponding BglII-NsiI fragment of Gα_{qi5} and Gα_{qs5/z5}, respectively. The correctness of all PCR-derived sequences was verified by sequencing in both directions.

Cell Culture and Transfections

COS7 cells were grown in Dulbecco's modified Eagles medium supplemented with 10% (vol/vol) fetal calf serum, Penicillin-Streptomycin (10000 IU/ml-10000 μg/ml) and 2 mM L-Glutamin in a humidified 5% CO₂ incubator. For transfections, 1×10⁶ cells were seeded into 100-mm dishes. About 24 h later, cells were cotransfected with the indicated G protein and receptor constructs (1:1 ratio) by using the Fugene transfection reagent as per manufacturer's instructions.

PI-Hydrolysis Assays

18-24 h after transfections cells were splitted into 35-mm dishes in culture medium supplemented with 3 μCi/ml [3H]myo-inositol (20 Ci/mmol; Amersham). After a 24 h labelling period cells were incubated during 20 min at room temperature with 2 ml of HANK's balanced salt solution containing 20 mM HEPES and 10 mM LiCl. Cells were then stimulated in the same buffer with the appropriate agonist ligands for 1 h (37° C.), and increases in intracellular inositolphosphate determined by anion exchange chromatography as described (Kostenis et al., 1997).

Western Blotting

COS7 cells were transfected in 100 mm dishes with 8 μg of G protein plasmid DNA using the Fugene transfection reagent according to the supplied protocol. For 8 μg of plasmid DNA 12 μl of Fugene were used. 72 h after transfection, cells were washed with ice cold PBS and harvested in a total volume of 2 ml PBS containing 1 mM phenylmethysulfonyl fluoride (PMSF). Cells were pelleted (5 min, 3000 rpm, Hettich EBA 12 centrifuge) and either stored at −80° C. or immediately processed to membranes. Cell pellets were resuspended in an ice cold buffer containing 50 mM Tris (pH 8), 2.5 mM MgCl₂, 1 mM EDTA, 1 mM PMSF (buffer A) and ruptured with 20 strokes of a hand-held glass homogenizer followed by passage (10 times) through a 27-gauge needle. Nuclei were pelleted (3500 rpm, 5 min, 4° C.) and the postnuclear supernatant was then fractionated (100.000 g, 30 min, 4° C.) into membrane pellets and supernatants. Pellets were then resuspended in buffer A. Membrane proteins were quantified with the Bio-Rad protein assay kit using bovine serum albumin (BSA) as a standard. Samples (20-40 μg of membrane protein) were resolved by SDS-polyacrylamide gel electrophoresis (13%), transferred to nitrocellulose membranes, and probed with the 12CA5-peroxidase linked monoclonal antibody (1:1000 dilution in TBS-T (10 mM Tris pH 8, 150 mM NaCl, 10% SDS, 0.1% Tween 20). Immunoreactive proteins were visualized with the enhanced chemiluminescence (ECL) kit from Amersham).

Receptor Ligands

Somatostatin 28 was from Bachem, (−)-quinpirole, carbachole, and U-50488 from Research Biochemicals Inc. (RBI), glucagone like peptide was Sigma, and glucose-dependent insulinotropic peptide was from Bachem. Chemerin9 was synthesized by UFpeptides (Ferrara, Italy) and the remaining chemicals were from Sigma.

Results and Discussion

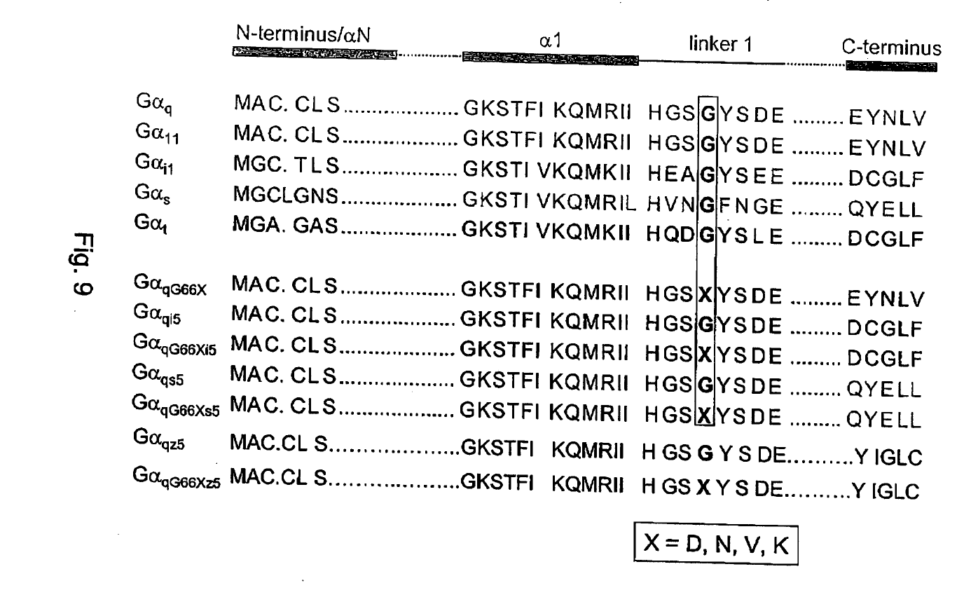

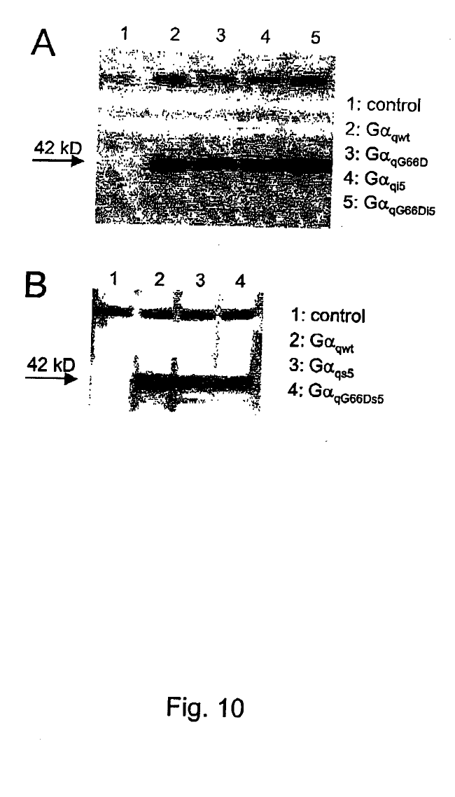

Replacement of the last 4-5 C-terminal amino acids of Gα_{q} with the corresponding Gαᵢ sequence (the resulting chimera is referred to as Gα_{qi5}) has been shown to confer onto Gi-linked receptors the ability to stimulate the Gq-pathway in assays measuring intracellular inositoltriphosphate (IP₃) generation or intracellular calcium mobilization (Conklin et al., 1993; Blahos et al., 1998; Kostenis et al., 1997). Similarly, Gs-coupled receptors can be forced to stimulate PLCβ upon coexpression with Gα_{qs5} (the last five C-terminal amino acids of Gα_{q} are replaced with the corresponding Gαₛ sequence) (Conklin et al., 1996). We have shown previously that mutation of a highly conserved glycine residue in linker I of the Gq subunit upon mutation to aspartate/valine/asparagines or lysine confer onto Gα_{q} the ability to channel Gi- and Gs-linked receptors to the PLCβ pathway. So far, no G protein α subunits have been created that combine the mutation in the linker I region and modification of the extreme C-terminus. In an attempt to create G protein α subunits with increased ability to link Gi- and Gs-coupled receptors to the Gq-pathway, we generated the following mutant Gα_{q}-constructs: (i) the highly conserved glycine in linker I is replaced by aspartate/valine/asparagines or lysine and (ii) the C-terminal 5 amino acids of Gα_{q} sequence were replaced by the corresponding Gαᵢ, Gαₛ, or Gα_{z} sequence, respectively. The mutants are referred to as Gα_{qG66Xi5}, Gα_{qG66Xs5} and Gα_{qG66Xz5} (X=D,V,N,K) and are schematically depicted in FIG. 9. The sequences of various wt Gα subunits as well as chimeric Gα_{qi5} and Gα_{s5/z5} are included in the alignment for means of comparison. Initially, expression levels of the mutant Gα subunits were compared with Gα_{q}wt, using membrane preparations of transiently transfected COS7 cells. All Gα subunits were detected with the monoclonal 12CA5-peroxidase linked antibody that recognizes an internal HA epitope tag. FIG. 10A indicates that both, wild type and mutant Gα_{q/i} subunits are expressed at similar levels. Likewise, the corresponding Gα_{q/s} mutant constructs are expressed at levels comparable to that of Gα_{q}wt (FIG. 10B).

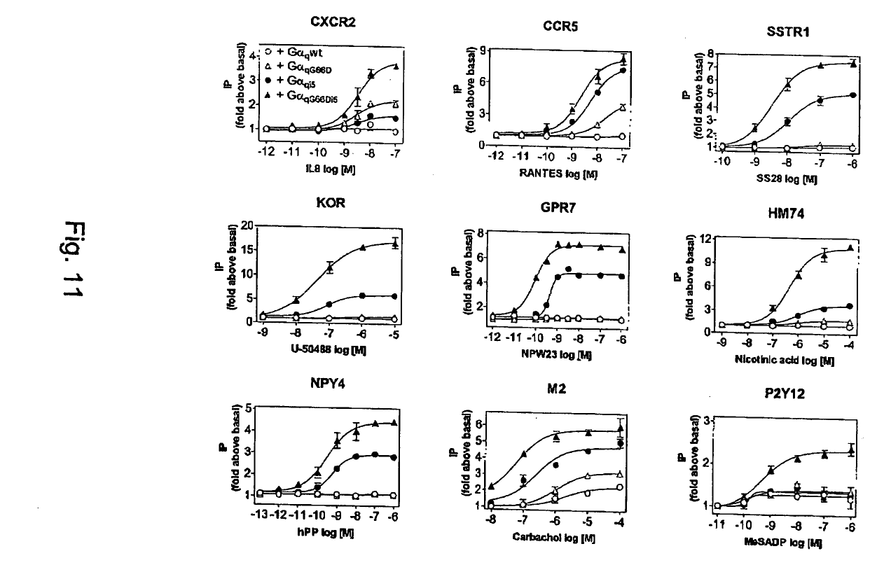

To characterize the functionality of the various mutant and chimeric Gα_{q/i} subunits, they were transiently coexpressed in COS7 cells with a series of Gi/o coupled receptors (chemokine CXCR2, chemokine CCR5, somatostatin receptor subtype 1 (SSTR1), kappa opioid receptor (KOR), GPR7, HM74a, neuropeptide Y receptor subtype 4 (NPY4), muscarinic M2, P2Y12)) and agonist induced mobilization of intracellular IP₃ was determined (FIG. 11). Coexpression of the different Gi/o coupled receptors with Gα_{q}wt resulted only in a rather small or no increase in PLCβ activity. Coexpression of the receptors with Gα_{qG66D} resulted in a significantly increased PI response (as compared with Gα_{q}wt) in case of the muscarinic M2, dopamine D2, CXCR2, and CCR5 receptor, but no IP₃ generation was observed for SSTR1. KOR, GPR7, HM74, NPY4, and P2Y12. In contrast, all except the P2Y12 receptor produced significant increases in inositolphosphate levels upon cotransfection with Gα_{qi5}. Interestingly, coexpression of all nine receptors with Gα_{qG66Di5} induced robust IP₃ increases exceeding those observed upon coexpression with the C-terminal or the G66D mutant alone.

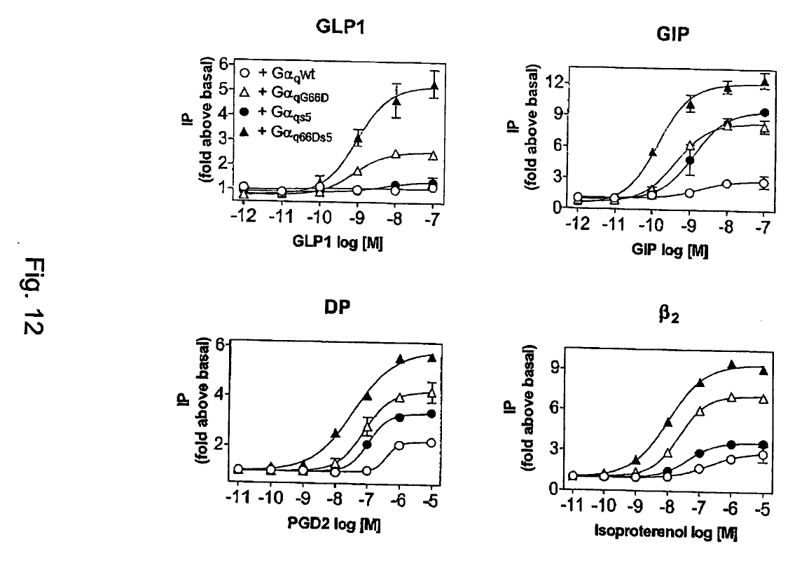

Likewise, intracellular IP₃ production was determined in COS7 cells cotransfected with the Gs-linked glucagon like peptide1 (GLP1) receptor, the glucose-dependent insulinotropic peptide (GIP) receptor, the prostaglandin D2 DP receptor, or the adrenergic β₂ receptor and vector DNA as a control or the various mutant Gα_{q} and Gα_{q/s} chimeric constructs (FIG. 12). As observed with the mutant and chimeric GaqA constructs, combination of the G66D mutation with the C-terminal sequence switch gave rise to a mutant Gα_{q/i} superior to the individual mutants with respect to stimulation of PLCβ.

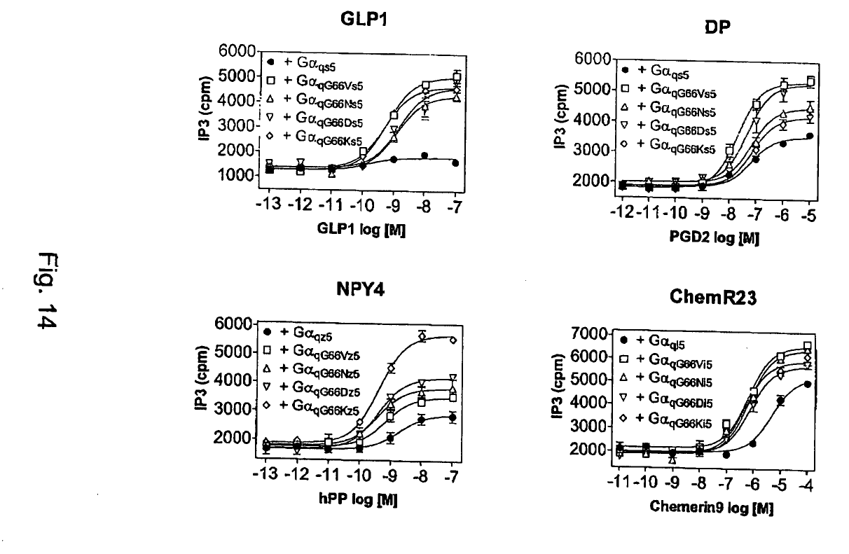

We also tested whether the cooperativity observed between the G66D mutation in linker I and the extreme C-terminus remains detectable when glycine is replaced by amino acids with different physicochemical properties (FIG. 14). To this end the GLP1, DP, NPY4, and ChemR23 receptor were cotransfected with various mutated Gα_{q} subunits and IP3 generation was determined as described in the experimental section. We observed that substitution of glycine by various other amino acids (aspartate, valine, asparagine, lysine) significantly potentiates the ability of C-terminally modified Gα proteins in linking receptors from various different coupling classes to the PLCβ pathway.

Collectively, these data provide evidence that both, the linker I region and The extreme C-terminus of Gα each determine receptor-G protein coupling specificity and that both regions act in concert in a cooperative manner to achieve maximal PLCβ stimulation by non-Gq-linked 7TM receptors

### REFERENCES

Bae, H., Anderson, K., Flood, L. A., Skiba, N P., Hamm, H. E., and Graber, S. G. (1997) J. Biol. Chem. 272, 32071-32077
Blahos, J.; Mary, S.: Perroy, J.; de Colle, C.; Brabet, I.; Bockaert, J.: Pin, J. P. Extreme C terminus of G protein alpha-subunits contains a site that discriminates between Gi-coupled metabotropic glutamate receptors. J. Biol. Chem. 1998, 273(40). 2576-25769.
Bockaert, J., and Pin, J.-P., (1999) EMBO J. 18, 1723-1729
Coleman, D E, Berghuis, A M, Lee, E., Linder M E, Gilman A G and Sprang, S R. Structure of active conformations of Gia1 and the mechanism of GTP hydrolysis. (1994) Science 265, 1405-1412
Conklin, B. R.; Farfel, Z.; Lustig, K. D.; Julius, D.; Bourne, H. R. Substitution of three amino acids switches receptor specificity of G_{q}α to that of Gᵢα. Nature 1993, 363, 274-276
Conklin, B. R.; Herzmark, P.; Ishida, S.; Voyno-Yasenetskaya, T. A.; Sun, Y.; Farfel, Z.; Bourne, H. R. Carboxyl-terminal mutations of Gq alpha and Gs alpha that alter the fidelity of receptor activation. Mol. Pharmacol. 1996, 50(4), 885-890
Ferguson, K M., Higashijima, T., Smigel, M. D., and Gilman, A. G. J. Biol. Chem. 261, 7393-7399,1986
Graziano M P, Gilman A G 1989 J. Biol. Chem 264, 15475-15482
Hamm, H. E., Deretic, D., Arendt, A., Hargrave, P. A., Koenig, B., and Hofmann, K. P. 1988) Science 241, 832-835
Iiri T, Herzmark P, Nakamoto J M, van Dop C, Bourne H R. Nature 1994 371, 164-168
Landis C A, Masters S B, Spada A, Pace A M, Bourne HR, Vallar L 1989 Nature 340,692-696
Iiri, T., Farfel, Z., and Bourne, H. R. Proc. Natl. Acad. Sci. USA, 5656-5661, 1997
Iiri, T., Farfel, Z., and Bourne, H. R. G protein diseases furnish a model for the turn-on switch. Nature 394, 35-38, 1998
Kostenis, E., Conklin, B. R., and Wess, J. Molecular basis of receptor/Gprotein coupling selectivity studied by coexpression of wild type and mutant m2 muscarinic receptors with mutant Gαq subunits. Biochemistry 36, 1487-1495, 1997
Kostenis, E. is Galpha16 the optimal tool for fishing ligands of orphan G-protein-coupled receptors? Trends Pharmacol Sci. 2001 November;22(11):560-4. Review.
Lambright, D. G., Noel, J. P., Hamm, H. E., and Sigler, P. B. Structural determinants for activation of the alpha-subunit of a heterotrimeric G protein Nature 369, 621-628, 1994
Lambright, D. G., Sondek, J., Bohm, A. Skiba, N. P., Hamm, H. E., and Sigler, P. B. The 2.0 Å crystal structure of a heterotrimeric G protein. Nature 379, 311-319,1996
Lee, E., Taussig, R., and Gilman, A. G. J. Biol. Chem. 267, 1212-1218, 1992
Lee, C. H., Park, D., Wu, D., Rhee, S. G., & Simon, M. I. Members of the Gqa subunit gene family activate phospholipase C b isozymes. J. Biol. Chem. 267, 16044-16047, 1992
Lee, C. H. et al. (1998) Differential coupling of G□q family of G-proteins to muscarinic M1 receptor and neurokinin-2 receptor. Arch. Pharm. Res. 4, 423-428
Marchese, A. et al. (1999) Novel GPCRs and their endogeneous ligands: expanding the boundaries of physiology and pharmacology. Trends Pharmacol. Sci. 20, 370-375
Masters S B, Miller R T, Chi M H, Chang F H, Beldermann B, Lopez N G, Bourne H R 1989 J. Biol. Chem 264, 15467-15474
Mazzoni, M. R., and Hamm, H. E. (1998) J. Biol. Chem. 271, 30034-30040
Milligan, G. et al. (1996) G₁₆ as a universal G protein adapter: implications for agonist screening strategies. Trends Pharmacol. Sci. 17, 235-237
Mixon, M. B., Lee. E., Coleman, D. E., Berghuis, A. M., Gilman, A. G., and Sprang, S. R. Tertiary and quaternary structural changes in Gi alpha 1 induced by GTP hydrolysis. Science, 270, 1995, 954-60.
Mody, S. M. et al. (2000) incorporation of Gaz-specific sequences at the carboxyl terminus increases the promiscuity of G□16 toward Gi-coupled receptors. Mol. Pharmacol. 57, 13-23
Noel, J P, Hamm, H E, Sigler P B, The 2.2 Å crystal structure of transducin-a complexed with GTPgS. Nature 1993,366,654-663
Offermanns, S. and Simon, M. I. Ga15 and Ga16 couple a wide variety of receptors to phosphollpase C. J. Biol. Chem. J. Biol. Chem., 270, 15175-15180, 1995
Onrust, R., Herzmark, P., Chi, P., Garcia, P. D., Lichtarge, O., Kingley, C., and Bourne, H. (1997) Science 275, 381-384
Ross, E. M., & Gilman, A. G. J. Biol. Chem. 252, 6966-6969
Seifert R, Wenzel-Selfert K, Lee T W, Gether U, Sanders-Bush E. Kobilka B. Different effects of Gsa splice variants on b2-adrenoceptor-mediated signalling. J. Biol. Chem. 273, 5109-5116,1998
Simon, M. I., Strathmann, M., Gautam, N. Diversity of G proteins in signal transduction. Science, 252, 802-808, 1991
Sondek, J., Bohm, A, Lambright, D. G., Hamm, H. E., and Sigler, P. B. Crystal structure of a G protein bg dimmer at 2.1 Å resolution. Nature 379, 369-374, 1996
Strader C D, Fong T M, Graziano M P, Tota M R. The family of G protein coupled receptors. FASEB J 1995, 9, 745-754, 1995
Strader C D, Fong T M, Tota M R, Underwood D, Dixon R A F. Structure and function of G protein coupled receptors. Annu Rev Biochem 1994, 63:101-132, 1994
Sprang, S. R. The structure of the G protein heterotrimer Gi alpha 1 beta 1 gamma 2. Cell, 83, 1047-1058, 1995.
Sunhara, R. K., Tesmer. J J G, Gilman, A G and Sprang S R. Crystal structure of the adenylyl cyclase activator Gsalpha. (1997) Science 278, 1943-1947
Wall, M. A., Coleman, D. E., Lee, E., Iniguez-Lluhi, J. A., Posner, B. A., Gilman, A. G., and Wedegaertner, P. S., Chu, I. H., Wilson, P. T., Levis, M. J., and Bourne, H. R. J. Biol. Chem. 268, 25001-25008, 1993
Wedegaertner, P. B.; Bourne, H. R. Activation and depalmitoylatlon of Gs alpha. Cell 1994, (77) 1063-1070

All documents mentioned herein are incorporated herein by reference in their entirety.

1. A q-type G protein comprising one or more mutations in the linker I and/or linker II region, the one or more mutations making the q-type G protein responsive to one or more non-q type G protein coupled receptors.

2. A q-type G protein according to claim 1, wherein the one or more mutations are deletions, substitutions and/or insertions.

3. A q-type G protein according to claim 1, wherein the one or more non-q type G protein coupled receptors are Gi type receptors.

4. A q-type G protein according to claim 1, wherein the one or more non-q type G protein coupled receptors are Gs type receptors.

5. A q-type G protein according to claim 1, wherein the one or more mutations are substitutions of at least one amino acid in the linker I region.

6. A q-type G protein according to claim 5, which is a Gα_{qG66X} mutant.

7. A q-type G protein according to claim 5, which is a Gα_{qG66X(X=D,N,V,K)} mutant.

8. A q-type G protein according to claim 1, which is Gα₁₁.

9. A q-type G protein according to claim 8, which is a Gα_{11G66X} mutant.

10. A q-type G protein according to claim 8, which is a Gα_{11 G66X(X=D,N,V,K)} mutant.

11. A q-type G protein according claim 1, which is Gα₁₄.

12. A q-type G protein according to claim 11, which is a Gα_{14G62X} mutant.

13. A q-type G protein according to claim 11, which is a Gα_{14 G62X(X=D,N,V,K)} mutant.

14. A q-type G protein according to claim 1, which is Gα₁₅.

15. A q-type G protein according to claim 14, which is a Gα_{15G69X} mutant.

16. A q-type G protein according to claim 14, which is a Gα_{15G69X(X=D,N,V,K)} mutant.

17. A q-type G protein according to claim 1, which is Gα₁₆.

18. A q-type G protein according to claim 17, which is a Gα_{16G69X} mutant.

19. A q-type G protein according to claim 17, which is a Gα_{16G69X(X=D,N,V,K)} mutant.

20. A q-type G protein according to claim 1, wherein the q-type G protein is further altered by replacing one or more amino acids at the C-terminus of the protein with the corresponding amino acids from an i-type G-protein.

21. A q-type G protein according to claim 20, wherein the last five amino acids at the C-terminus are replaced with the corresponding amino acids from an i-type G-protein.

22. A q-type G protein according to claim 1, wherein the q-type G protein is further altered by replacing one or more amino acids at the C-terminus of the protein with the corresponding amino acids from a s-type G-protein.

23. A q-type G protein according to claim 22, wherein the last five amino acids at the C-terminus are replaced with the corresponding amino acids from a s-type G-protein.

24. A q-type G protein according to claim 31 having SEQ ID No. 1 (Gα_{qG66X(X=D,N,V,K)} mutant).

25. A q-type G protein according to claim 24 comprising an amino acid sequence having at least about 75% such as at least about 80%, at least about 85%, at least about 90% or at least about 95% identity to the amino acid sequence of SEQ ID No. 1.

26. A q-type G protein according to claim 1 having SEQ ID No. 2 (Gα_{11G66X(X=D,N,V,K)} mutant).

27. A q-type G protein according to claim 26 comprising an amino acid sequence having at least about 75% such as at least about 80%, at least about 85%, at least about 90% or at least about 95% identity to the amino acid sequence of SEQ ID No. 2.

28. A q-type G protein according to claim 1 having SEQ ID No. 3 (Gα_{144G62X(X=D,N,V,K)} mutant).

29. A q-type G protein according to claim 28 comprising an amino acid sequence having at least about 75% such as at least about 80%, at least about 85%, at least about 90% or at least about 95% identity to the amino acid sequence of SEQ ID No. 3.

30. A q-type G protein according to claim 1 having SEQ ID No. 4 (Gα_{15G69X(X=D,N,V,K)} mutant).

31. A q-type G protein according to claim 30 comprising an amino acid sequence having at least about 75% such as at least about 80%, at least about 85%, at least about 90% or at least about 95% identity to the amino acid sequence of SEQ ID No. 4.

32. A q-type G protein according to claim 1 having SEQ ID No. 5 (Gα_{16G69X(X=D,N,V,K)} mutant).

33. A q-type G protein according to claim 32 comprising an amino acid sequence having at least about 75% such as at least about 80%, at least about 85%, at least about 90% or at least about 95% identity to the amino acid sequence of SEQ ID No. 5.

34. A q-type G protein according to claim Th having SEQ ID No. 6 (Gα_{qG66X(X=D,N,V,K)ii5}).

35. A q-type G protein according to claim 34 comprising an amino acid sequence having at least about 75% such as at least about 80%, at least about 85%, at least about 90% or at least about 95% identity to the amino acid sequence of SEQ ID No. 6.

36. A q-type G protein according to claim 1 having SEQ ID No. 7 (Gα_{qG66X(X=D,N,V,K)s5}).

37. A q-type G protein according to claim 36 comprising an amino acid sequence having at least about 75% such as at least about 80%, at least about 85%, at least about 90% or at least about 95% identity to the amino acid sequence of SEQ ID No. 7.

38. An isolated nucleic acid molecule comprising a polynucleotide encoding a q-type G protein according to claim 1.

39. A recombinant DNA expression vector comprising a nucleic acid molecule of claim 38.

40. A host cell transformed with the vector according to claim 39.

41. A method for identifying modulators of a G-protein coupled receptor in a sample, comprising the steps of
a) contacting a cell comprising one or more G-protein coupled receptors and one or more q-type G proteins according to clam 1 with the sample,
b) detecting any q-type G protein response.

42. A method according to claim 41, wherein the G-protein coupled receptor is a non-Gq type receptor.

43. A method according to claim 41, wherein the G-protein coupled receptor is a Gi-type receptor.

44. A method according to claim 43, wherein the Gi-receptor is selected from the biogenic amine family, peptide receptors, and chemokine receptor.

45. A method according to claim 41, wherein the G-protein coupled receptor is a Gs-type receptor.

46. A method according to claim 45, wherein the Gs-receptor is selected from the biogenic amine receptors and family B receptors.

47. A method according to claim 41, wherein the G-protein coupled receptor is an orphan receptor.

48. A method according to claim 41, wherein the detection comprises measuring phospholipase C, hydrolysis of phosphatidylinositols and/or calcium mobilization.

49. A method according to claim 48, wherein the detection comprises measuring of calcium mobilization.

50. A high-throughput screening method comprising a method according to claim 41.

51. A method according to claim 41, wherein the modulator is an antagonist, a partial antagonist, an inverse antagonist, an agonist, a partial agonist, an inverse agonist, an allosteric enhancer or an allosteric inhibitor.

52. A method according to claim 41 for identifying one or more lead compounds for use in drug discovery methods.

53. A method according to claim 41 for identifying one or more lead compounds for use in target validation methods.

54. A method according to claim 41 for identifying one or more lead compounds for use in a chemogenomic model.

55. A method according to claim 41 for identifying a compound for use in medicine.

56. A compound identifiable by a method according to claim 41.

57. A compound for use in a drug discovery method, in a target validation method, in a chemogenomic model and/or in medicine identifiable by a method according to claim 41.

58. A pharmaceutical composition comprising a compound for use in medicine identifiable by a method according to claim 41 together with a pharmaceutically acceptable excipient.


SEQUENCE LISTING (7 sequences)

SEQ ID NO: 1 - protein, 359 aa, Mus musculus
  MISC_FEATURE (66)..(66): Xaa is selected from the group consisting of Asp, Val, Asn and Lys
MTLESIMACCLSEEAKEARRINDEIERHVRRDKRDARRELKLLLLGTGESGKSTFIKQMRIIHGSXYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPSYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 2 - protein, 359 aa, Mus musculus
  MISC_FEATURE (66)..(66): Xaa is selected from the group consisting of Asp, Val, Asn and Lys
MTLESMMACCLSDEVKESKRINAEIEKQLRRDKRDARRELKLLLLGTGESGKSTFIKQMRIIHGAXYSEEDKRGFTKLVYQNIFTAMQAMVRAMETLKILYKYEQNKANALLIREVDVEKVTTFEHQYVNAIKTLWSDPGVQECYDRRREFQLSDSAKYYLTDVDRIATVGYLPTQQDVLRVRVPTTGIIEYPFDLENIIFRMVDVGGQRSERRKWIHCFENVTSIMFLVALSEYDQVLVESDNENRMEESKALFRTIITYPWFQNSSVILFLNKKDLLEDKILHSHLVDYFPEFDGPQRDAQAAREFILKMFVDLNPDSDKIIYSHFTCATDTENIRFVFAAVKDTILQLNLKEYNLV

SEQ ID NO: 3 - protein, 355 aa, Mus musculus
  MISC_FEATURE (62)..(62): Xaa is selected from the group consisting of Asp, Val, Asn and Lys
MAGCCCLSAEEKESQRISAEIERQLRRDKKDARRELKLLLLGTGESGKSTFIKQMRIIHGSXYSDEDRKGFTKLVYQNIFTAMQAMIRAMDTLRIQYVCEQNKENAQIIREVEVDKVSMLSREQVEAIKQLWQDPGIQECYDRRREYQLSDSAKYYLTDIDRIATPSFVPTQQDVLRVRVPTTGIIEYPFDLENIIFRMVDVGGQRSERRKWIHCFESVTSIIFLVALSEYDQVLAECDNENRMEESKALFKTIITYPWFLNSSVILFLNKKDLLEEKIMYSHLISYFPEYTGPKQDVRAARDFILKLYQDQNPDKEKVIYSHFTCATDTDNIRFVFAAVKDTILQLNLREFNLV

SEQ ID NO: 4 - protein, 374 aa, Mus musculus
  MISC_FEATURE (69)..(69): Xaa is selected from the group consisting of Asp, Val, Asn and Lys
MARSLTWGCCPWCLTEEEKTAARIDQEINRILLEQKKQEREELKLLLLGPGESGKSTFIKQMRIIHGVXYSEEDRRAFRLLIYQNIFVSMQAMIDAMDRLQIPFSRPDSKQHASLVMTQDPYKVSTFEKPYAVAMQYLWRDAGIRACYERRREFHLLDSAVYYLSHLERISEDSYIPTAQDVLRSRMPTTGINEYCFSVKKTKLRIVDVGGQRSERRKWIHCFENVIALIYLASLSEYDQCLEENDQENRMEESLALFSTILELPWFKSTSVILFLNKTDILEDKIHTSHLATYFPSFQGPRRDAEAAKSFILDMYARVYASCAEPQDGGRKGSRARRFFAHFTCATDTQSVRSVFKDVRDSVLARYLDEINLL

SEQ ID NO: 5 - protein, 374 aa, Homo sapiens
  MISC_FEATURE (69)..(69): Xaa is selected from the group consisting of Asp, Val, Asn and Lys
MARSLTWRCCPWCLTEDEKAAARVDQEINRILLEQKKQDRGELKLLLLGPGESGKSTFIKQMRIIHGAXYSEEERKGFRPLVYQNIFVSMRAMIEAMERLQIPFSRPESKHHASLVMSQDPYKVTTFEKRYAAAMQWLWRDAGIRACYERRREFHLLDSAVYYLSHLERITEEGYVPTAQDVLRSRMPTTGINEYCFSVQKTNLRIVDVGGQKSERKKWIHCFENVIALIYLASLSEYDQCLEENNQENRMKESLALFGTILELPWFKSTSVILFLNKTDILEEKIPTSHLATYFPSFQGPKQDAEAAKRFILDMYTRMYTGCVDGPEGSKKGARSRRLFSHYTCATDTQNIRKVFKDVRDSVLARYLDEINLL

SEQ ID NO: 6 - protein, 359 aa, Mus musculus
  MISC_FEATURE (66)..(66): Xaa is selected from the group consisting of Asp, Val, Asn and Lys
MTLESIMACCLSEEAKEARRINDEIERHVRRDKRDARRELKLLLLGTGESGKSTFIKQMRIIHGSXYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPSYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKDCGLF

SEQ ID NO: 7 - protein, 359 aa, Mus musculus
  MISC_FEATURE (66)..(66): Xaa is selected from the group consisting of Asp, Val, Asn and Lys
MTLESIMACCLSEEAKEARRINDEIERHVRRDKRDARRELKLLLLGTGESGKSTFIKQMRIIHGSXYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPSYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKQYELL